(19) United States
(12) Patent Application Publication
(10) Pub. No.: US 2023/0248696 A1
(43) Pub. Date: Aug. 10, 2023

(54) LENALIDOMIDE PRODRUGS, POLYMERIC CONJUGATES, AND FORMULATIONS THEREOF, AND THEIR USES FOR THE TREATMENT OF MULTIPLE MYELOMA

(71) Applicant: DYNAMIC BIOLOGICS INC., Monmouth Junction, NJ (US)

(72) Inventors: Deven Patel, Cedar Grove, NJ (US); H. Rajan Sharma, Belle Mead, NJ (US); Manoj Mishra, Woodstock, MD (US); Lemma Reddy PEDDAREDDYGARI, Monroe, NJ (US)

(21) Appl. No.: 18/004,665

(22) PCT Filed: May 3, 2020
(86) PCT No.: PCT/US2020/031238

§ 371 Date: Jan. 6, 2023

Related U.S. Application Data
(60) Provisional application No. 62/842,658, filed on May 3, 2019.

(51) Int. Cl. A61K 31/404; A61K 9/00; A61K 47/44; A61K 9/14; A61K 47/60

(52) U.S. Cl. CPC A61K 31/404; A61K 9/0019; A61K 47/44; A61K 9/14; A61K 47/60

(57) ABSTRACT

Compounds of formula I and II:

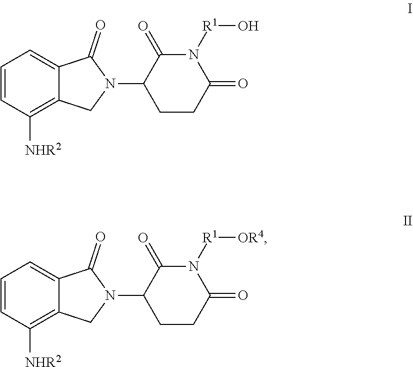

or a pharmaceutically acceptable salt thereof, and their compositions including polymer encapsulated micro/nano particle compositions are provided, wherein:
R¹ is —CH₂—, a C₂₋₆ straight or branched chain alkylene, alkenylene or alkynylene group or a short chain polyethylene glycol group having 2-6 monomers, or a combination thereof;
R² is hydrogen or —(C═O)R³ wherein R³ is a C2-6 straight or branched chain alkylene, alkenylene or alkynylene group or a short chain polyethylene glycol group having 2-6 monomers, or a combination thereof; and
R⁴ is a pharmaceutically acceptable polymeric moiety comprising a pharmaceutically acceptable polymer chain such that R¹ is linked to the polymer chain through an ester, carbonate or carbamate bond including the oxygen atom linking R¹ and R⁴.

The compositions are useful for treatment of multiple myeloma, mantle cell lymphoma, and transfusion-dependent anemia due to myelodysplastic syndromes.

### CROSS REFERENCE TO RELATED PATENT APPLICATION

This application claims priority to provisional U.S. Application No. 62/842,658 filed May 3, 2019, the contents of which are herein incorporated by reference in their entirety.

### FIELD OF INVENTION

The present invention relates to prodrugs of lenalidomide, polymer conjugates of the prodrugs and polymeric nanoparticle/microparticle formulations thereof that are useful for their anti-tumor and immunomodulatory effects and angiogenesis inhibition.

### BACKGROUND OF THE INVENTION

Lenalidomide (Brand name Revlimid®) in combination with dexamethasone is currently approved in the US for the treatment of multiple myeloma, as well as for maintenance therapy of multiple myeloma following autologous hematopoietic stem cell transplantation.

Lenalidomide is also approved for the treatment of mantle cell lymphoma in patients whose disease has relapsed or progressed after two prior therapies, one of which included bortezomib. Lenalidomide is also approved for the treatment of patients with transfusion-dependent anemia due to low- or intermediate-1-risk myelodysplastic syndromes (MDS) associated with a deletion 5q cytogenetic abnormality with or without additional cytogenetic abnormalities.

The mechanism of action of lenalidomide is not fully known but it has been demonstrated that lenalidomide increases production of anti-inflammatory cytokine IL-10, IL-2 and Interferon gamma (IFN-γ) from peripheral blood mononuclear cells and is a potent inhibitor of pro-inflammatory cytokines TNF-α, IL-1β, IL-6 and IL-12. It also inhibits cyclooxygenase-2 (COX-2) but not COX-1 expression. Lenalidomide shows direct anti-tumor effect, inhibits angiogenesis and has immunomodulatory effect in vitro. In vivo, it induces tumor cell apoptosis directly and indirectly by inhibition of bone marrow stromal cell support, by antiangiogenic and anti-osteoclastogenic effects, and by immunomodulatory activity.

At the molecular level, lenalidomide has been shown to interact with the ubiquitin E3 ligase cereblon and targets this enzyme to degrade the Ikaros transcription factors IKZF1 and IKZF3. This mechanism was unexpected as it suggests that the major action of lenalidomide is to re-target the activity of an enzyme rather than block the activity of an enzyme or signaling process, and thereby represents a novel mode of drug action. Lenalidomide inhibits cell proliferation with varying effectiveness in some but not all cell lines. Lenalidomide is also effective in inhibiting growth of Namalwa cells but is much less effective in inhibiting growth of KG-1 cells and other cell lines without chromosome 5 deletions.

Lenalidomide is available in the form of capsules with multiple strengths (2.5, 5, 10, 15, 20 and 25 mg). The initial starting dose is 10 mg orally once a day and the dosage is adjusted based on renal clearance of the drug. Under fasting conditions >90% of oral lenalidomide dose is absorbed rapidly. However, food affects oral absorption, reducing area under the concentration-time curve (AUC) by 20% and maximum concentration (Cmax) by 50%. On oral administration, peak Cmax is seen in 1 hour. Lenalidomide has a short half-life of 3 to 4 hours and does not accumulate in plasma upon repeated dosing. Lenalidomide is the predominant circulating component in humans and does not undergo metabolism via cytochrome P450 pathway. However, there are two identified metabolites, hydroxy-lenalidomide and N-acetyl-lenalidomide; each constitutes less than 5% of parent levels in circulation. Lenalidomide is cleared primarily by renal excretion.

Lenalidomide is a BCS class 3 (high solubility and low permeability) substance. Upon oral administration of 10 mg once a day, the maximum plasma concentration (Cmax) of lenalidomide achieved is between 190 and 210 ng/mL. The therapeutic Cmax range of lenalidomide is between 50 and 540 ng/mL when the dose is 5 to 25 mg/day. Co-administration with food does not alter the extent of absorption (AUC) but does reduce the maximal plasma concentration (Cmax) by 36%. The pharmacokinetic disposition of lenalidomide is linear. The most frequently reported adverse events were hematological (neutropenia, thrombocytopenia and anemia) and gastrointestinal (diarrhea, constipation, nausea, vomiting, abdominal pain and dry mouth). The most common serious adverse events were hematological, general (pyrexia, asthenia) respiratory (pleural effusion, dyspnea, infection, cardiac disorders (failure, atrial fibrillation), vascular (deep vein thrombosis) and gastrointestinal (diarrhea). Dyspepsia is seen in 20% of multiple myeloma patients treated with lenalidomide and neurotoxicity causes 75% of patients to discontinue maintenance therapy.

Lenalidomide is a thalidomide analogue having the chemical name, 3-(4′ aminoisoindoline-1′-one)-1-piperidine-2, 6-dione. It is a synthetic derivative of glutamic acid and is structurally very close to thalidomide. Although it is a chiral molecule and possesses an asymmetric carbon, it has been developed as a racemic mixture because it undergoes racemization under physiological conditions. Lenalidomide exhibits polymorphism but is commercially prepared in a hemihydrate form, which is non-hygroscopic. CTP-221, a deuterated S— lenalidomide analog, was found to be more potent than lenalidomide in key biological activities related to clinical efficacy and is under development by Concert Pharmaceuticals. It has been found to have greater potency compared to racemic lenalidomide for IL-2 induction (increased production), TNF-alpha inhibition and anti-proliferative activities in vitro.

### SUMMARY OF THE INVENTION

The invention provides certain prodrugs of lenalidomide and their polymer conjugates with linear, branched and globular biocompatible polymers. These compounds offer sustained-release properties compared to free lenalidomide that is known to cause side effects depending on dosage. The invention also provides nanoparticle/microparticle formulations of lenalidomide prodrugs and their polymer conjugates using biocompatible pharmaceutically acceptable polymers. The compounds and compositions of the invention provide improved bioavailability and reduce the frequency of dosing and total dosage of lenalidomide, thereby improving the side effect profile of lenalidomide.

In some embodiments, the present invention provides a prodrug compound of formula I:

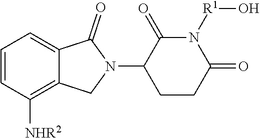

or a pharmaceutically acceptable salt thereof, wherein:
R¹ is —CH₂—, a C₂₋₆ straight or branched chain alkylene, alkenylene or alkynylene group or a short chain polyethylene glycol group having 2-6 monomers, or a combination thereof;
and
R² is hydrogen or —(C═O)R³ wherein R³ is a C2-6 straight or branched chain alkylene, alkenylene or alkynylene group or a short chain polyethylene glycol group having 2-6 monomers, or a combination thereof.

In other embodiments, the present invention provides a polymer conjugate compound of formula II:

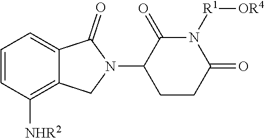

or a pharmaceutically acceptable salt thereof, wherein:
R¹ is —CH₂—, a C₂₋₆ straight or branched chain alkylene, alkenylene or alkynylene group or a short chain polyethylene glycol group having 2-6 monomers, or a combination thereof;
R² is hydrogen or —(C═O)R³ wherein R³ is a C2-6 straight or branched chain alkylene, alkenylene or alkynylene group or a short chain polyethylene glycol group having 2-6 monomers, or a combination thereof; and
R⁴ is a pharmaceutically acceptable polymeric moiety comprising a pharmaceutically acceptable polymer chain such that R¹ is linked to the polymer chain through an ester, carbonate or carbamate bond including the oxygen atom linking R¹ and R⁴.

In certain other embodiments, the present invention provides a polymer conjugate compound of formula III:

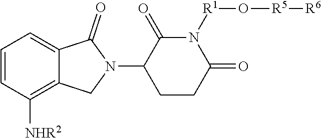

or a pharmaceutically acceptable salt thereof, wherein:
R¹ is —CH₂—, a C₂₋₆ straight or branched chain alkylene, alkenylene or alkynylene group or a short chain polyethylene glycol group having 2-6 monomers, or a combination thereof;
R² is hydrogen or —(C═O)R³ wherein R³ is a C2-6 straight or branched chain alkylene, alkenylene or alkynylene group or a short chain polyethylene glycol group having 2-6 monomers, or a combination thereof;
R⁵ is a linker selected from —(C═O)R⁷—(C═O)O—; —(C═O)R⁷—(C═O)N—; —(C═O)R⁷—O(C═O)O—; —(C═O)R⁷—O(C═O)NH—; —(C═O)R⁷—NH(C═O)O—; —(C═O)OR⁷—(C═O)O—; —(C═O)OR⁷—(C═O)N—; —(C═O)OR⁷—O(C═O)O—; —(C═O)OR⁷—O(C═O)NH—; —(C═O)OR⁷—NH(C═O)O—; —(C═O)NHR⁷—(C═O)O—; —(C═O)NHR⁷—(C═O)N—; —(C═O)NHR⁷—O(C═O)O—; —(C═O)NHR⁷—O(C═O)NH—; —(C═O)NHR⁷—NH(C═O)O—;
R⁷ is —CH₂—, a C₂₋₆ straight or branched chain alkylene, alkenylene or alkynylene group or a short chain polyethylene glycol group having 2-6 monomers, or a combination thereof; and
R⁶ is a pharmaceutically acceptable polymeric moiety comprising a pharmaceutically acceptable polymer chain.

Yet other embodiments of the present invention provide a pharmaceutical composition wherein the composition comprises micro or nano particles comprising:

the compound of formula I, II or III; and
a second pharmaceutically acceptable polymer,
wherein the compound of formula I, II or III is encapsulated in the second pharmaceutically acceptable polymer.

Pharmaceutically acceptable polymers used in the present invention may be linear, branched or globular.

In some embodiments of the invention the pharmaceutically acceptable polymer and/or the second pharmaceutically acceptable polymer is independently selected from the group consisting of polyethylene glycol (PEG), poly(glycolide) (PGA), poly(lactide) (PLA), poly(caprolactone), poly(lactide-co-caprolactone), poly(lactide-co-glycolide) (PLGA), and poly(lactic acid)-butanol, poly(vinyl pyrrolidone), poly(vinyl alcohol) (PVA), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof. In some embodiments of the invention the pharmaceutically acceptable polymer and/or the second pharmaceutically acceptable polymer used for encapsulation is selected from the group consisting of PLA, PLGA, PVA, and combinations thereof in different proportions.

Certain embodiments of the invention provide compositions comprising a pharmaceutically effective amount of the compound formula I, II or III and one or more pharmaceutically acceptable carriers or excipients. In particular, castor oil or its derivatives may be used as an excipient. In some embodiments, the compositions are in the form of liposomes or micelles using pharmaceutically acceptable ampiphillic compounds.

In certain embodiments of the invention, the micro or nano particles compositions comprising polymer encapsulated compound of formula I, II or III further comprise one or more pharmaceutically acceptable carriers or excipients.

The compositions of the invention are useful for their anti-tumor and immunomodulatory effects and angiogenesis inhibition, such as for treatment of cancer including for the treatment of multiple myeloma, mantle cell lymphoma, or transfusion-dependent anemia due to myelodysplastic syndromes. In particular, the compositions are useful for the same treatments as lenalidomide, such as the approved indications of lenalidomide.

In some embodiments, the compositions of the invention may be administered parenterally, such as intravenously, intramuscularly, or subcutaneously. In certain other embodiments, the compositions of the invention may be administered topically, such as in the form of transdermal patches, creams, foams, gels, lotions, ointments, sprays, and eye drops that are applied epicutaneously, applied to the conjunctiva or through inhalation.

In some embodiments, the compositions of the invention may be administered at most twice weekly. For example, the compositions of the invention may be administered once weekly, biweekly, or once monthly.

The compositions of the invention offer improved chemical and pharmaceutical properties, such as superior pharmacokinetic properties, compared to lenalidomide and require substantially reduced dosage to achieve therapeutic plasma concentration due to their structure and mode of administration. The compositions of the invention reduce adverse events and variability in pharmacokinetics, in part by avoiding local high concentrations of the drug within the gastrointestinal tract upon oral administration and reducing gastrointestinal side effects.

### DETAILED DESCRIPTION

Pharmaceutically acceptable polymers used in the present invention may be non-toxic, non-immunogenic, non-antigenic, highly soluble in water and FDA (The Food and Drug Administration) approved. The polymer conjugate compounds of the invention and polymer-encapsulated compounds of the invention have several advantages: a prolonged residence in body, a decreased degradation by metabolic enzymes and a reduction or elimination of protein immunogenicity. The covalent attachment of polymer to a drug can increase its hydrodynamic size (size in solution), which prolongs its circulatory time by reducing renal clearance (Knop et al., Angew. Chemie Int. Ed. 2010; 49(36):6288-6308; Veronese et al., Drug Discov Today. 2005; 10(21):1451-1458; and Harris et al., Nat Rev Drug Discov. 2003; 2(3):214-221). Advantages of pharmaceutical compositions disclosed herein include, for example: increased bioavailability at lower doses; predictable drug-release profile over a defined period of time following each administration; better patient compliance; ease of application; improved systemic availability by avoidance of first-pass metabolism; reduced dosing frequency without compromising the effectiveness of the treatment; decreased incidence of side effects; and overall cost reduction of medical care.

In some embodiments, R¹ is —CH₂—, R² is hydrogen in the compound of formula I. In other embodiments, R¹ is —CH₂—, R² is hydrogen, and R⁴ is a pharmaceutically acceptable polymeric moiety comprising straight, branched or globular chain polyethylene glycol in the compound of formula II.

Polymer conjugates of formula II and III may be prepared by methods known in the art, for example, Sk U H et al., Biomacromolecules. 2013; 14(3):801-10. Polymer-encapsulated micro/nano particles may be prepared by methods known in the art. For example, Han et al., Front Pharmacol. 2016; 7:185; Qutachi O et al., Acta Biomater. 2014; 10(12):5090-5098.

In some embodiments, the pharmaceutically acceptable polymer in compounds of formula II or III comprises 15-75 monomer units, 20-70 monomer units, or 25-65 monomer units. In other embodiments, the polymer has a molecular weight in the range of 1 kDa to 75 kDa, 2 kDa to 60 kDa, or 3 kDa to 50 kDa.

In certain other embodiments, the pharmaceutically acceptable polymer in compounds of formula II or III is a branched chain PEG comprising 4-120 monomer units, 4-75 monomer units, 4-50 monomer units, or 4-30 monomer units. In certain other embodiments, the polymer is a straight or branched chain PEG comprising 12-120 monomer units, 12-75 monomer units, 12-75 monomer units, or 12-30 monomer units. In some other embodiments, the polymer is a straight or branched chain PEG comprising 11-20 monomer units, 26-42 monomer units, 49-64 monomer units, or 72-111 monomer units. In certain other embodiments, the polymer is a straight or branched chain PEG having a molecular weight in the range of 0.4 kDa to 50 kDa, 0.5 kDa to 50 kDa, 0.8 kDa to 50 kDa, or 1 kDa to 50 kDa.

The term “encapsulated” in the context of the present invention means coated by, covered by, or surrounded by, such that about 20% to about 80% of the compound of formula I or II is enclosed/covered/coated by the polymer.

In some embodiments, PLGA and mixture of PLGA with other polymers, such as PLA and PVA, in different ratios are used to encapsulate compounds of the invention to form microparticles. PLGA, is a pharmaceutically acceptable widely used biodegradable material use for encapsulation of a broad range of therapeutic agents including hydrophilic and hydrophobic small molecule drugs, DNA, and proteins, due to its excellent biocompatibility. Other additives can be used to enhance the drug loading and efficiency in PLGA microparticles, such as PEG, poly(orthoesters), chitosan, alginate, caffeic acid, hyaluronic acid etc. PLGA can be a varying composition of PLA and PGA with a ratio from 20 to 80% PGA in PLA and vice versa.

In some embodiments, the amount of compound of formula I, II or III in the compositions of the invention is in the range of 1 mg to 50 mg equivalent of lenalidomide. In some other embodiments, the amount of compounds compound of formula I or II in the compositions of the invention is in the range of 2.5 mg to 25 mg equivalent of lenalidomide.

In some embodiments, dosage forms of the composition of the invention are adapted for administration to a patient parenterally, including subcutaneous, intramuscular, intraperitoneal, intravenous or intradermal injections. In other embodiments, the composition may be administered as a depot.

Upon parenteral injection of lenalidomide-polymer conjugates of formula II or III, enzymatic cleavage may occur generating the compound of formula I, and the respective polymer used in the conjugation. A water-mediated hydrolysis may convert compound of formula I to the active form, lenalidomide, and formaldehyde. When R² is —(C═O)R³′ enzymatic hydrolysis may occur simultaneously to generate lenalidomide, and a carboxylic acid R³COOH.

In some embodiment, the compositions of the invention further comprise one or more pharmaceutically effective carriers or excipients. Pharmaceutical compositions adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents.

The compositions may be presented in unit-dose or multi-dose containers, for example, sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

### Examples

a) Preparation of N-hydroxymethyl lenalidomide

Dissolve lenalidomide in DMF (dimethyl formamide) and stir the reaction under nitrogen atmosphere for 10 minutes. Add thiethylamine followed by formaldehyde in DMF and stir the reaction mixture at 80° C. for 24 hours. After completion of the reaction, evaporate all solvents and purify the crude mixture using column chromatography. Characterize the resulting N-hydroxymethyl lenalidomide using reverse-phase HPLC, proton NMR and mass spectroscopic techniques.

b) Preparation of Lenalidomide-polymer conjugates

Dissolve carboxylate functionalized linear/branched PEG or any other carboxylate functionalized globular polymer in anhydrous DMF under nitrogen atmosphere. Add EDC and DMAP to the reaction mixture and dissolve in DMF. Stir the reaction mixture for 30 minutes. Add a calculated amount of N-hydroxymethyl lenalidomide dissolved in DMF to the reaction mixture and stir the reaction mixture for 2 days under nitrogen atmosphere. Evaporate the solvent and dialyze the resulting reaction mixture for 24 h with water using dialysis membrane (MWCO 1 kDa). Lyophilize the resulting water to get the final lenalidomide-polymer conjugates. Check the purity of the conjugate by Reverse-phase HPLC and characterize/calculate the loading of the polymeric conjugate by proton NMR, and MALDI-TOF mass spectroscopy.

c) Preparation of Lenalidomide Microparticle

Nanoprecipitation technique is used for the preparation of the lenalidomide microparticles. Briefly, lenalidomide and polymer (e.g., PLGA) are dissolved in a suitable solvent (e.g., dichlromethane) in different ratios, the mixture being subjected to sonication for 5-10 minutes to achieve dissolution, if required. Dissolve a hydrophilic non-ionic surfactant (for example a triblock copolymer), such as Pluronic F127, in 50 mL of deionized water and add the lenalidomide/PLGA solution dropwise using a syringe with a flow rate of 1 mL/10 min with stirring at varying speed. Centrifuge, and lyophilize the obtained nanosuspension with cryoprotectant (e.g., 2% sucrose). Characterize the microparticle with scanning electron microscopy (SEM), differential scanning calorimetry (DSC) and X-Ray diffraction (XRD).

Other compound of formula I, II and III may be prepared by similar procedures using conventional methods known in the art. Lenalidomide may be prepared by methods known in the art or obtained from commercial sources.

1. A compound of formula: 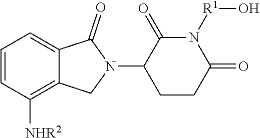
or a pharmaceutically acceptable salt thereof, wherein:
R¹ is —CH₂—, a C₂₋₆ straight or branched chain alkylene, alkenylene or alkynylene group or a short chain polyethylene glycol group having 2-6 monomers, or a combination thereof; and
R² is hydrogen or —(C=0)R³ wherein R³ is a C2-6 straight or branched chain alkylene, alkenylene or alkynylene group or a short chain polyethylene glycol group having 2-6 monomers, or a combination thereof.

2. A compound of formula II: 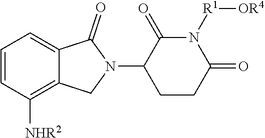
or a pharmaceutically acceptable salt thereof, wherein:
R¹ is —CH2-, a C₂₋₆ straight or branched chain alkylene, alkenylene or alkynylene group or a short chain polyethylene glycol group having 2-6 monomers, or a combination thereof;
R² is hydrogen or —(C=0)R³ wherein R³ is a C2-6 straight or branched chain alkylene, alkenylene or alkynylene group or a short chain polyethylene glycol group having 2-6 monomers, or a combination thereof; and
R⁴ is a pharmaceutically acceptable polymeric moiety comprising a pharmaceutically acceptable polymer chain such that R¹ is linked to the polymer chain through an ester, carbonate or carbamate bond including the oxygen atom linking R¹ and R⁴.

3. A compound of formula III: 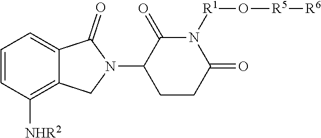
or a pharmaceutically acceptable salt thereof, wherein;
R¹ is —CH₂—; a C₂₋₆ straight or branched chain alkylene, alkenylene or alkynylene group or a short chain polyethylene glycol group having 2-6 monomers, or a combination thereof;
R² is hydrogen or —(C=0)R³ wherein R³ is a C₂₋₆ straight or branched chain alkylene, alkenylene or alkynylene group or a short chain polyethylene glycol group having 2-6 monomers, or a combination thereof;
R⁵ is a linker selected from —(C=0)R⁷—(C=0)0-; —(C=0)R⁷—(C=0)N—;
—(C=0)R⁷-0(C=0)0-; —(C═O) R⁷-0 (C=0)NH—; —(C=0)R⁷—NH(C=0)0-;
—(C=0)0R⁷—(C=0)0-; —(C=0)0R⁷—(C=0)N—; —(C=0)0R⁷-0(C=0)0-;
—(C=0)0R⁷-0(C=0)NH—; —(C=0)0R⁷—NH(C=0)0-; —(C=0)NHR⁷—(C=0)0-;
—(C=0)NHR⁷—(C=0)N—; —(C=0)NHR⁷-0(C=0)0-; —(C=0)NHR⁷-0(C=0)NH—;
—(C=0)NHR⁷—NH(C=0)0-;
R⁷ is —CH₂—, a C₂₋₆ straight or branched chain alkylene, alkenylene or alkynylene group or a short chain polyethylene glycol group having 2-6 monomers, or a combination thereof; and
R⁶ is a pharmaceutically acceptable polymeric moiety comprising a pharmaceutically acceptable polymer chain.

4. The compound of claim 2, wherein the pharmaceutically acceptable polymer chain comprising the polymeric moiety R⁴ is selected from the group consisting of polyethylene glycol, poly(glycolide), poly(lactide), poly(caprolactone), poly(lactide-co-caprolactone), poly(lactide-co-glycolide), and poly(lactic acid)-butanol, poly(vinyl pyrrolidone), poly(vinyl alcohol), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof.

5. The compound of claim 3, wherein the pharmaceutically acceptable polymeric moiety R⁶ is selected from the group consisting of polyethylene glycol, poly(glycolide), poly(lactide), poly(caprolactone), poly(lactide-co-caprolactone), poly(lactide-co-glycolide), and poly(lactic acid)-butanol, poly(vinyl pyrrolidone), poly(vinyl alcohol), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof.

6. A composition comprising a pharmaceutically effective amount of the compound of claim 1 and one or more pharmaceutically acceptable carriers or excipients.

7. The composition of claim 6, wherein the composition is injectable or topical.

8. (canceled)

9. The composition according to claim 6, wherein the pharmaceutically acceptable carrier is castor oil or a derivative thereof.

10. A pharmaceutical composition comprising a pharmaceutically effective amount of the compound of claim 1, wherein the composition comprises micro or nano particles comprising:
the compound of formula I; and
a second pharmaceutically acceptable polymer,
wherein the compound of formula I is encapsulated in the second pharmaceutically acceptable polymer.

11. The pharmaceutical composition according to claim 10, wherein the second pharmaceutically acceptable polymer is selected from the group consisting of polyethylene glycol, poly(glycolide), poly(lactide), poly(caprolactone), poly(lactide-co-caprolactone), poly(lactide-co-glycolide), and poly(lactic acid)-butanol, poly(vinyl pyrrolidone), poly(vinyl alcohol), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof.

12. The pharmaceutical composition according to claim 10, wherein the composition further comprises one or more pharmaceutically acceptable carriers or excipients.

13. The composition of claim 12, wherein the composition is injectable or topical.

14. A method for the treatment of multiple myeloma, mantle cell lymphoma, or transfusion-dependent anemia due to myelodysplastic syndromes in a subject in need thereof comprising administering a composition according to claim 6 to the subject.

15. The method according to claim 14, wherein the composition is administered intravenously, intramuscularly, intraperitoneally, or subcutaneously.

16. The method according to claim 15, wherein the composition is administered once monthly, biweekly, once weekly, or at most twice weekly.

17. (canceled)

18. (canceled)

19. (canceled)

20. A method for the treatment of multiple myeloma, mantle cell lymphoma, or transfusion-dependent anemia due to myelodysplastic syndromes in a subject in need thereof comprising administering a composition according to claim 12 to the subject.

21. The method according to claim 20, wherein the composition is administered intravenously, intramuscularly, intraperitoneally, or subcutaneously.

22. The method according to claim 21, wherein the composition is administered once monthly, biweekly, once weekly, or at most twice weekly.

23. (canceled)

24. (canceled)

25. (canceled)

26. A pharmaceutical composition comprising a pharmaceutically effective amount of the compound of claim 2, wherein the composition comprises micro or nano particles comprising:
the compound of formula II; and
a second pharmaceutically acceptable polymer,
wherein the compound of formula II is encapsulated in the second pharmaceutically acceptable polymer.

27. A pharmaceutical composition comprising a pharmaceutically effective amount of the compound of claim 3, wherein the composition comprises micro or nano particles comprising:
the compound of formula III; and
a second pharmaceutically acceptable polymer,
wherein the compound of formula III is encapsulated in the second pharmaceutically acceptable polymer.
